(19) United States
(12) Patent Application Publication
(10) Pub. No.: US 2020/0061542 A1
(43) Pub. Date: Feb. 27, 2020

(54) METHODS FOR IMPROVING FILTERABILITY OF POLYSACCHARIDE-PROTEIN CONJUGATE REACTIONS

(71) Applicant: Merck Sharp & Dohme Corp., Rahway, NJ (US)

(72) Inventors: Michael A. Winters, Doylestown, PA (US); Patrick McHugh, Pipersville, PA (US); Kyle Evans, Royersford, PA (US); Jian He, Blue Bell, PA (US); Josef Vlasak, Schwenksville, PA (US); Willam J. Smith, Harleysville, PA (US)

(73) Assignee: Merck Sharp & Dohme Corp., Rahway, NJ (US)

(21) Appl. No.: 16/487,628

(22) PCT Filed: Feb. 20, 2018
(86) PCT No.: PCT/US2018/018658

§ 371 Date: Aug. 21, 2019

Related U.S. Application Data
(60) Provisional application No. 62/463,229, filed on Feb. 24, 2017.

(51) Int. Cl. B01D 61/14; A61K 39/09; B01D 71/10

(52) U.S. Cl. CPC B01D 61/145; A61K 39/092; A61K 2039/6037; B01D 61/147; B01D 71/10

(57) ABSTRACT

The present invention provides improved methods for preparing polysaccharide protein conjugates utilizing surfactants to improve filterability of the conjugation reaction mixture. The polysaccharide-protein conjugates are useful for inclusion in vaccines.

### FIELD OF INVENTION

The present invention provides improved methods for preparing polysaccharide protein conjugates utilizing surfactants to improve filterability of the conjugates, improve stability, and prevent aggregate formation. The polysaccharide-protein conjugates are useful for inclusion in vaccines.

### BACKGROUND OF THE INVENTION

Polysaccharide (Ps)-protein conjugate vaccines, comprising bacterial capsular polysaccharides conjugated to carrier proteins have been developed and are commercially available. Examples of such conjugate vaccines include the Haemophilus influenzae type b (Hib) conjugate vaccine (e.g., HIBTITER®) as well as conjugate vaccines against Streptococcus pneumoniae (e.g., PREVNAR® and PREVNAR® 13) and Neisseria meningitidis (e.g., MENJUGATE® and MENVEO®).

Various methods for the purification of Ps-protein conjugates are known in the art, including hydrophobic chromatography, tangential ultrafiltration, diafiltration, etc. See, e.g., International Patent Application Publication No. WO00/38711, U.S. Pat. No. 6,146,902, and Lei et al., 2000, Dev. Biol. 103:259-264. Upon the conjugation of a polysaccharide antigen to a carrier protein, the reaction mixture is typically purified with one or more purification steps (to enrich for polysaccharide protein conjugates) before a final filtration using a 0.2-micron filter to sterilize the drug substance prior to formulation of the drug product.

Larger Ps-protein conjugates have been shown in the literature to be more immunogenic than smaller conjugates (See, e.g., Lee et al., 2009, Vaccine 27(5):726-732). It is therefore desirable to produce larger conjugates. However, the ability of the commonly used 0.2-micron filtration to filter Ps-protein conjugate becomes more challenging as conjugate size increases. This is due to filter fouling in which larger conjugates are more prone to block pores within the 0.2-micron filter than smaller conjugates. As a result, there is often significant yield loss of larger conjugate particles and inconsistency between different runs. Ps-protein conjugates may also aggregate during processing due to hydrophobic interactions between conjugated proteins.

Accordingly, there is a continuing need for improved methods of filtering and purifying Ps-protein conjugates and increasing their stability.

### SUMMARY OF THE INVENTION

The present invention provides improved methods for preparing polysaccharide protein conjugates utilizing surfactants to improve filterability of the conjugates and prevent aggregate formation. In the methods of the invention, a surfactant is present during the sterile filtration of the conjugate reaction prior to formulating into a vaccine.

Accordingly, the present invention provides methods for purifying a polysaccharide protein conjugate, the method comprising: a) reacting a polysaccharide with a protein to form a polysaccharide protein conjugate reaction mixture; b) performing purification of the polysaccharide protein conjugate reaction mixture by ultrafiltration, diafiltration or column chromatography; and c) performing a sterile filtration of the polysaccharide protein conjugate reaction mixture in the presence of a surfactant. The surfactant can be added at any step of the reaction/process so long as surfactant is present during the sterile filtration, i.e., the surfactant is carried through to the last step. In some embodiments, the surfactant is added during step c). In some embodiments, the surfactant is added during step b). In some embodiments, the surfactant is added during step a).

In certain embodiments, the sterile filtration is by a 0.2-0.45 μm filter, preferably using a sterilizing or bioburden reduction filtration with a 0.2 μm or 0.22 μm filter.

In certain embodiments, the methods of the invention result in improved filterability, for example, as measured by mass of the conjugate filtered per membrane area and/or increased conjugate size. In certain aspects, the mass of the conjugate filtered per membrane area or average conjugate size (as measured by HPSEC/UV/MALS/RI) is improved by 10%, 20%, 30%, 40%, 50%, 60%, 70%, 80%, 90%, or 100% or more compared to a control without surfactant. In certain aspects, the mass of the conjugate filtered per membrane area is improved by 2-fold, 3-fold, 5-fold or more compared to a control without surfactant. In certain aspects, the average conjugate size (as measured by HPSEC/UV/MALS/RI) is improved by 1.2-fold, 1.5-fold or 2-fold or more compared to a control without surfactant.

In certain embodiments, the surfactant is an ionic surfactant, a nonionic surfactant or a zwitterionic surfactant. In one embodiment, the zwitterionic surfactant is CHAPS. In certain embodiments, the surfactant is a nonionic surfactant. In one aspect of this embodiment, the surfactant is PS-20 present at a concentration range between 0.007% to 0.5% (w/v).

The purification in step b) can be performed by any known method for purification, including column chromatography, ultrafilation, diafiltration, etc. In one aspect, the purification is by ultrafiltration or diafiltration. In certain aspects, the ultrafiltration or diafiltration is performed with a 1 to 1,000 kDa, 50 to 1,000 kDa or 100 to 1,000 kDa, 300 to 500 kDa MWCO (Molecular Weight Cut Off) membrane made of regenerated cellulose. In one aspect, the membrane is a 300 kDa MWCO membrane made of regenerated cellulose.

In certain embodiments, the polysaccharide is selected from the group consisting of Meningococcal polysaccharides, Pneumococcal polysaccharides, Hemophilus influenzae type b polysaccharide, Vi polysaccharide of Salmonnella typhi, and group B Streptococcus polysaccharides. In certain aspects of these embodiments, the polysaccharide is a pneumococcal polysaccharide.

In certain embodiments, the protein is selected from the group consisting of tetanus toxoid, diphtheria toxoid, and CRM₁₉₇. In one aspect of this embodiment, the protein is CRM₁₉₇.

In certain embodiments, the reacting is by reductive amination. In one aspect, the reductive amination is performed under aqueous conditions. In another aspect, the reductive amination is performed in dimethylsulfoxide (DMSO).

In certain aspects, the reductive amination comprises: a) reacting a polysaccharide with an oxidizing agent, whereby a solution of an aldehyde-activated polysaccharide is obtained; b) adjusting the pH of the solution of the aldehyde-activated polysaccharide to a pH of from 4 to 7, if necessary; and c) reacting the activated polysaccharide with a protein in the presence of a reducing agent to form a polysaccharide protein conjugate. Where reductive amination is performed in DMSO, it is the reacting step c) which includes DMSO. Where reductive amination is performed in an aqueous solvent, the aqueous solvent in reacting step c) is at a pH of from 6 to 7.5. In certain aspects, the solution of the aldehyde-activated polysaccharide is buffer exchanged with a buffer containing acetate or phosphate. In certain aspects, the oxidizing agent is sodium periodate (NaIO₄). In certain aspects, the reducing agent is sodium cyanoborohydride (NaCNBH₃) or sodium borohydride (NaBH₄).

For polysaccharides from certain serotypes, it is beneficial to react the polysaccharide protein conjugate with a strong reducing agent to reduce unreacted groups. In certain aspects, the strong reducing agent is NaBH₄.

### BRIEF DESCRIPTION OF THE DRAWINGS

FIG. 1: Filterability as measured by pressure increase as a function of filter loading. Two serotype 5-CRM₁₉₇ conjugates (lots A and B) were filtered at constant flow rate each through the same type of 0.2-micron filter. Inlet pressure was recorded as a function of the mass of conjugated Ps per m² of filter area. Lot A did not contain PS-20. Lot B contained PS-20. Prior to filtration, the conjugate size of lot B was 21% larger than lot A.

FIG. 2: Filterability as measured by pressure increase as a function of filter loading. Two serotype 6A-CRM₁₉₇ conjugates (lots A and B) were filtered at constant flow rate through a 0.2-micron filter. Inlet pressure was recorded as a function of the mass of conjugated Ps per m² of filter area. Lot A did not contain PS-20. Lot B contained PS-20. Prior to filtration, the conjugate size of lot B was 34% larger than lot A.

FIGS. 3A-B: Vortexing impact on the aggregate profile of Pneumococcal Ps-protein conjugate. 23F-CRM₁₉₇ conjugate as prepared in Example 3 was used in this study. The presence of aggregates was measured by flow cytometry. The samples were evaluated before and after vortexing, without PS-20 (A) or after spiking with 0.05% (w/v) final concentration of PS-20 (B). X-axis indicates side-light scatter signal in arbitrary units that is proportional to size and refractive index of the particle. The vertical dashed line indicates the limit of detection of the flow cytometer.

FIG. 4: The impact of vortexing on aggregate formation for Pneumococcal Ps-CRM₁₉₇ conjugates prepared in aqueous solution versus in dimethylsulfoxide (DMSO). Concentration of detectable particles (open bars) or the mean light scattering (LS) value of the particles (solid bars) expressed as a ratio between the vortexed and untreated sample is shown. Conjugates made in aqueous buffer were prepared as per Example 2. Conjugates made in DMSO were prepared as per Example 3.

FIGS. 5A-B: Surfactant screening with serotype 19A (A) and 23F (B). Concentration of detectable particles (open bars) and the mean light scattering (LS) value of the particles (solid bars) are expressed as a fold change compared to a control sample with no surfactant (first set of bars). Conjugates reacted in DMSO were prepared as per Example 3.

### DETAILED DESCRIPTION OF THE INVENTION

The present invention provides improved methods for preparing polysaccharide protein conjugates utilizing surfactants to improve filterability of the conjugation reaction mixture and the stability of the conjugates. More specifically, the present invention provides a method for purifying a polysaccharide protein conjugate by filtering a polysaccharide protein conjugate reaction mixture with a sterilizing grade filter in the presence of a surfactant. The surfactant can be added at any point during the purification steps compatible with the functionality of a surfactant.

As used herein, the term “polysaccharide” is meant to include any antigenic saccharide element (or antigenic unit) commonly used in the immunologic and bacterial vaccine arts, including, but not limited to, a “saccharide”, an “oligosaccharide”, a “polysaccharide”, a “liposaccharide”, a “lipo-oligosaccharide (LOS)”, a “lipopolysaccharide (LPS)”, a “glycosylate”, a “glycoconjugate” and the like.

As used herein, the term “comprises” when used with the immunogenic composition of the invention refers to the inclusion of any other components (subject to limitations of “consisting of” language for the antigen mixture), such as adjuvants and excipients. The term “consisting of” when used with the multivalent polysaccharide-protein conjugate mixture refers to a mixture having those particular S. pneumoniae polysaccharide protein conjugates and no other S. pneumoniae polysaccharide protein conjugates from a different serotype.

As used herein, the phrase “drug product” refers to the formulated blend of polysaccharide-carrier protein conjugates from one or more serotypes.

As used herein, the phrase “drug substance” refers to the individual polysaccharide-carrier protein conjugate from a given serotype.

As defined herein, “improved filterability” refers to an increase in filter loading, usually occurring before a pressure rise or decay in filtration flow rate, or an increase in average conjugate size (due to a reduction in filter fouling). Filter loading is measured by the mass of conjugate filtered (often measured by polysaccharide) per membrane area through standard techniques. Conjugate size is typically measured by HPSEC/UV/MALS/RI.

As defined herein, the terms “precipitation”, “precipitate” “particulate formation”, “clouding” and “aggregation” may be used interchangeably and are meant to refer to any physical interaction or chemical reaction which results in the “aggregation” of a polysaccharide-protein conjugate. The process of aggregation (e.g., protein aggregation) is often influenced by numerous physicochemical stresses, including heat, pressure, pH, agitation, shear forces, freeze-thawing, dehydration, heavy metals, phenolic compounds, silicon oil, denaturants and the like.

As defined herein, a “surfactant” of the present invention is any molecule or compound that is amphiphillic (having both a hydrophilic and hydrophobic structure) and lowers the surface tension of the medium. The surfactant could also posseses the properties of a detergent, wetting agent, emulsifier and the like. Surfactants are classified based on the inherent ability to dissociate in water and include nonionic, anionic, cationic or zwitterionic properties. A “surfactant system” comprises a surfactant but may allow for the inclusion of additional excipients such as polyols, osmolytes that increase the effects of the surfactant.

As used herein, the term “ultrafiltration” refers to membrane filtration in which forces like pressure or concentration gradients lead to a separation through a semipermeable membrane. Suspended solids and solutes of high molecular weight are retained in the so-called retentate, while water and low molecular weight solutes and molecules pass through the membrane in the permeate (filtrate). Ultrafiltration membranes are typically less than or equal to 1000 kDa MWCO. Ultrafiltration can be distinguished from microfiltration which generally uses a wider pore size, e.g., 0.1 μm, having a higher MWCO.

As used herein, the term “diafiltration” refers to a type of ultrafiltration involving a buffer exchange that involves removal or separation of components of a solution based on their molecular size by using permeable filters in order to obtain pure solution. The membrane used for diafiltration is typically referred to as an ultrafiltration membrane.

As used herein, all ranges, for example, pH, temperature, and concentrations, are meant to be inclusive. For example, a pH range from 5.0 to 9.0 is meant to include a pH of 5.0 and a pH of 9.0. Similarly, a temperature range from 4 to 25° C. is meant to include the outer limits of the range, i.e., 4° C. and 25° C.

### Polysaccharide

Bacterial capsular polysaccharides are suitable for use in the methods of the invention and can readily be identified using known methods. These bacterial capsular polysaccharides may for example be from N. meningitidis, particularly serogroups A, C, W135 and Y; S. pneumoniae, particularly from serotypes: 1, 2, 3, 4, 5, 6A, 6B, 6C, 6D, 7B, 7C, 7F, 8, 9N, 9V, 10A, 11A, 12F, 14, 15A, 15B, 15C, 16F, 17F, 18C, 19A, 19F, 20, 21, 22A, 22F, 23A, 23B, 23F, 24F, 27, 28A, 31, 33F, 34, 35A, 35B, 35F, and 38; S. agalactiae, particularly serotypes Ia, Ib, and III; S. aureus, particularly from S. aureus type 5 and type 8; Haemophilus influenzae Type b; Salmonella enterica Typhi Vi; and Clostridium difficile. The invention may also use non-capsular bacterial polysaccharides. An exemplary non-capsular bacterial polysaccharide is the S. pyogenes GAS carbohydrate (also known as the GAS cell wall polysaccharide, or GASP). The invention may also use non-bacterial polysaccharides. For example, the invention may use glucans, e.g. from fungal cell walls. Representative glucans include laminarin and curdlan.

The polysaccharides may be used in the form of oligosaccharides. These are conveniently formed by fragmentation of purified polysaccharide (e.g. by hydrolysis), which will usually be followed by purification of the fragments of the desired size.

Polysaccharides can be purified by known techniques. The invention is not limited to polysaccharides purified from natural sources, however, and the polysaccharides may be obtained by other methods, such as total or partial synthesis. Capsular polysaccharides from Streptococcus pneumoniae can be prepared by standard techniques known to those skilled in the art. For example, polysaccharides can be isolated from bacteria and may be sized to some degree by known methods (see, e.g., European Patent Nos. EP497524 and EP497525); and preferably by microfluidisation accomplished using a homogenizer or by chemical hydrolysis. Polysaccharides can be sized in order to reduce viscosity in polysaccharide samples and/or to improve filterability for conjugated products. In one embodiment, each pneumococcal polysaccharide serotype is grown in a soy-based medium. The individual polysaccharides are then purified through standard steps including centrifugation, precipitation, and ultra-filtration. See, e.g., U.S. Patent Application Publication No. 2008/0286838 and U.S. Pat. No. 5,847,112. Polysaccharides can be sized in order to reduce viscosity in polysaccharide samples and/or to improve filterability for conjugated products.

The purified polysaccharides can be chemically activated to make the saccharides capable of reacting with the carrier protein. The purified polysaccharides can be connected to a linker. Once activated or connected to a linker, each capsular polysaccharide is separately conjugated to a carrier protein to form a glycoconjugate. The polysaccharide conjugates may be prepared by known coupling techniques including reductive amination, carbodiimide (e.g. EDAC or EDC) chemistry, 1-cyano-4-dimethylamino Pyridinium tetrafluoroborate (CDAP) chemistry, hydrazides, active esters, norborane, p-nitrobenzoic acid, N-hydroxysuccinimide, S-NHS, and TSTU. See, for example, International Patent Application Publication No. WO98/42721.

In certain embodiments, the polysaccharide can be coupled to a linker to form a polysaccharide-linker intermediate in which the free terminus of the linker is an ester group. The linker is therefore one in which at least one terminus is an ester group. The other terminus is selected so that it can react with the polysaccharide or activated polysaccharide to form the polysaccharide-linker intermediate.

In certain embodiments, the polysaccharide can be coupled to a linker using a primary amine group in the polysaccharide. In this case, the linker typically has an ester group at both termini. This allows the coupling to take place by reacting one of the ester groups with the primary amine group in the polysaccharide by nucleophilic acyl substitution. The reaction results in a polysaccharide-linker intermediate in which the polysaccharide is coupled to the linker via an amide linkage. The linker is therefore a bifunctional linker that provides a first ester group for reacting with the primary amine group in the polysaccharide and a second ester group for reacting with the primary amine group in the carrier molecule. A typical linker is adipic acid N-hydroxysuccinimide diester (SIDEA).

The coupling can also take place indirectly, i.e. with an additional linker that is used to derivatise the polysaccharide prior to coupling to the linker. The polysaccharide can be coupled to the additional linker using a carbonyl group at the reducing terminus of the polysaccharide. This coupling comprises two steps: (a1) reacting the carbonyl group with the additional linker; and (a2) reacting the free terminus of the additional linker with the linker. In these embodiments, the additional linker typically has a primary amine group at both termini, thereby allowing step (a1) to take place by reacting one of the primary amine groups with the carbonyl group in the polysaccharide by reductive amination. A primary amine group is used that is reactive with the carbonyl group in the polysaccharide. Hydrazide or hydroxylamino groups are suitable. The same primary amine group is typically present at both termini of the additional linker. The reaction results in a polysaccharide-additional linker intermediate in which the polysaccharide is coupled to the additional linker via a C—N linkage.

The polysaccharide can be indirectly coupled to the additional linker using a different group in the polysaccharide, particularly a carboxyl group. This coupling comprises two steps: (a1) reacting the group with the additional linker; and (a2) reacting the free terminus of the additional linker with the linker. In this case, the additional linker typically has a primary amine group at both termini, thereby allowing step (a1) to take place by reacting one of the primary amine groups with the carboxyl group in the polysaccharide by EDAC activation. A primary amine group is used that is reactive with the EDAC-activated carboxyl group in the polysaccharide. A hydrazide group is suitable. The same primary amine group is typically present at both termini of the additional linker. The reaction results in a polysaccharide-additional linker intermediate in which the polysaccharide is coupled to the additional linker via an amide linkage.

In one embodiment, the chemical activation of the polysaccharides and subsequent conjugation to the carrier protein are achieved by means described in U.S. Pat. Nos. 4,365,170, 4,673,574 and 4,902,506. Briefly, that chemistry entails the activation of pneumococcal polysaccharide by reaction with any oxidizing agent which oxidizes a terminal hydroxyl group to an aldehyde, such as periodate (including sodium periodate, potassium periodate, or periodic acid). The reaction leads to a random oxidative cleavage of vicinal hydroxyl groups of the carbohydrates with the formation of reactive aldehyde groups.

In this embodiment, coupling to the protein carrier can be by reductive amination via direct amination to the lysyl groups of the protein. For example, conjugation is carried out by reacting a mixture of the activated polysaccharide and carrier protein with a reducing agent such as sodium cyanoborohydride, optionally in the presence of nickel. The conjugation reaction may take place under aqueous conditions or in the presence of dimethylsulfoxide (DMSO). See, e.g., U.S. Patent Application Publication Nos. US2015/0231270 A1 and US2011/0195086 A1, and European Patent No. EP 0471 177 B1. Unreacted aldehydes are then optionally capped with the addition of a strong reducing agent, such as sodium borohydride. For example, serotype 5 from S. pneumoniae does not require capping.

In certain embodiments, the conjugation reaction is performed by reductive amination wherein nickel is used for greater conjugation reaction efficiency and to aid in free cyanide removal. Transition metals are known form stable complexes with cyanide and are known to improve reductive methylation of protein amino groups and formaldehyde with sodium cyanoborohydride (Gidley et al., 1982, Biochem J. 203: 331-334; Jentoft et al. 1980, Anal Biochem. 106: 186-190). By complexing residual, interfering cyanide, the addition of nickel increases the consumption of protein during the conjugation of and leads to formation of larger, potentially more immungenic conjugates.

In another embodiment, the conjugation method may rely on activation of the saccharide with 1-cyano-4-dimethylamino pyridinium tetrafluoroborate (CDAP) to form a cyanate ester. The activated saccharide may thus be coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using GMBS) or a haloacetylated carrier protein (for example using iodoacetimide [e.g. ethyl iodoacetimide HCl] or N-succinimidyl bromoacetate or SIAB, or SIA, or SBAP). Preferably, the cyanate ester (optionally made by CDAP chemistry) is coupled with hexane diamine or adipic acid dihydrazide (ADH) and the amino-derivatised saccharide is conjugated to the carrier protein using carbodiimide (e.g. EDAC or EDC) chemistry via a carboxyl group on the protein carrier. Such conjugates are described in International Patent Application Publication Nos. WO 93/15760, WO 95/08348 and WO 96/29094; and Chu et al., 1983, Infect. Immunity 40:245-256.

Other suitable techniques use carbodiimides, hydrazides, active esters, norborane, p-nitrobenzoic acid, N-hydroxysuccinimide, S-NHS, EDC, TSTU. Many are described in International Patent Application Publication No. WO 98/42721. Conjugation may involve a carbonyl linker which may be formed by reaction of a free hydroxyl group of the saccharide with CDI (See Bethell et al., 1979, J. Biol. Chem. 254:2572-4; Hearn et al., 1981, J. Chromatogr. 218:509-18) followed by reaction of with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

After conjugation of the capsular polysaccharide to the carrier protein, the polysaccharide-protein conjugates are subjected to purification (enriched with respect to the amount of polysaccharide-protein conjugate) by one or more of a variety of techniques. Examples of these techniques are well known to the skilled artisan and include concentration/diafiltration operations, ultrafiltration, precipitation/elution, column chromatography, and depth filtration. See, e.g., U.S. Pat. No. 6,146,902.

Applicants have also discovered that the type of filter can make a significant difference in the filterability of the conjugate, particularly during ultrafiltration/diafiltration. Specifically, improved results were seen using a filter of regenerated cellulose compared to a filter of polyethersulfone. This could be due to non-specific binding of surfactant in a protein containing formulation to PES and PVDF membranes. See, e.g., Zhou et al. 2008, J Memb Sci 325:735-741.

Regenerated cellulose membranes suitable for ultrafiltration/diafiltration are available from commercial sources such as Corning Inc. (Tewksbury, Mass.), GE Healthcare Bio-Sciences (Pittsburgh, Pa.), EMD Millipore (Burlington, Mass.), Sartorius Corp. (Bohemia, N.Y.).

In embodiments where a final reduction step (e.g., capping free aldehydes) is performed, purification can be performed after each step or after a single step.

After an initial purification, the polysaccharide protein conjugation reaction mixture is subjected to sterile filtration typically using a 0.2-0.45 micron filter. Prior to the work described herein, filterability of the polysaccharide protein conjugate was potentially an issue, particularly with high molecular weight conjugates.

Accordingly, in the methods of the invention, a surfactant is included prior to a final filtration step (to reduce bioburden and sterilize the resulting drug substance) to improve filterability. The surfactant can be added at any step in the process where the surfactant is compatible with the solvent(s) being used. In conjugate reactions performed entirely under aqueous conditions, a surfactant can be added at any step in the process. For example, it can be added just prior to the final filtration step, it can be added prior to the initial purification step, or it can be included in the reaction mixture. In conjugation reactions performed in the presence of an organic solvent, such as DMSO, the surfactant is generally not added during the reaction, but it can be added before the reaction or it can be added subsequent to DMSO removal from the reaction mixture (DMSO removal by means such as ultrafiltration/diafiltration). Alternatively, the DMSO levels can be reduced to below about 20% or less with a diluent containing surfactant.

Suitable surfactants for use in the methods of the invention include ionic surfactants, anionic surfactants and zwitterionic surfactants.

Ionic surfactants include deoxycholate, sodium dodecyl sulfate, and the copolymers of ethylene oxide (EO), propylene oxide (PO), and/or butylene oxide (BO), sold under the DOWFAX™ tradename, such as linear EO/PO block copolymers.

Non-ionic surfactants include polyoxyethylene sorbitan ester surfactants (commonly referred to as the Tweens), especially PS-20 and PS-80; polyoxyethylene fatty ethers derived from lauryl, cetyl, stearyl and oleyl alcohols (known as Brij surfactants), such as triethyleneglycol monolauryl ether (Brij 30); octoxynols, which can vary in the number of repeating ethoxy (oxy-1,2-ethanediyl) groups, with octoxynol-9 (Triton X-100, or t-octylphenoxypolyethoxyethanol) being of particular interest; Mega-10; nonionic triblock copolymer composed of a central hydrophobic chain of polyoxypropylene (poly(propylene oxide)) flanked by two hydrophilic chains of polyoxyethylene (poly(ethylene oxide)) (known as poloxamers or under the tradename)Pluronic®; nonylphenol ethoxylates, such as the Tergitol™ NP series; (octylphenoxy)polyethoxyethanol (IGEPAL CA-630/NP-40; and sorbitan esters (commonly known as the SPANs), such as sorbitan trioleate (Span 85) and sorbitan monolaurate.

Zwitterionic surfactants include CHAPS and Zwittergent 3-10. In certain embodiments, the surfactant is not Zwittergent 3-10.

Surfactants which can be either anionic or non-ionic include copolymers of ethylene oxide (EO), propylene oxide (PO), and/or butylene oxide (BO), sold under the DOWFAX™ tradename, such as linear EO/PO block copolymers.

Surfactants which can be anionic or zwitterionic include various phospholipids such as phosphatidylcholine (lecithin).

Surfactants are amphilic molecules, possessing both hydrophiliic and hydrophobic properties. As a consequence of this dual chemical property, a surfactant generally orients itself so as to minimize exposure of its hydrophobic portion to the aqueous medium. This can be at solid surfaces (e.g. vessels, syringes, vials), biotherapeutic surfaces (hydrophobic patches on proteins) or in micelles. As a surfactant concentration increases, it approaches a critical limit whereby the monomeric surfactants aggregate into micelles and is referred to as the critical micelle concentration (CMC). Generally, a surfactant could stabilize biotherapeutics against aggregation at levels below, at or above the CMC.

A preferred surfactant is PS-20, which is used at above its CMC of 0.007% (w/v). Accordingly, PS-20 can be used from 0.007% to about 0.5% (w/v), preferably between 0.01% to 0.2% (w/v).

### Carrier Protein

In a particular embodiment of the present invention, CRM₁₉₇ is used as the carrier protein. CRM₁₉₇ is a non-toxic variant (i.e., toxoid) of diphtheria toxin. In one embodiment, it is isolated from cultures of Corynebacterium diphtheria strain C7 (β197) grown in casamino acids and yeast extract-based medium. In another embodiment, CRM₁₉₇ is prepared recombinantly in accordance with the methods described in U.S. Pat. No. 5,614,382. Typically, CRM₁₉₇ is purified through a combination of ultra-filtration, ammonium sulfate precipitation, and ion-exchange chromatography. In some embodiments, CRM₁₉₇ is prepared in Pseudomonas fluorescens using Pfenex Expression Technology™ (Pfenex Inc., San Diego, Calif.).

Other suitable carrier proteins include additional inactivated bacterial toxins such as DT (Diphtheria toxoid), TT (tetanus toxid) or fragment C of TT, pertussis toxoid, cholera toxoid (e.g., as described in International Patent Application Publication No. WO 2004/083251), E. coli LT (heat-labile enterotoxin), E. coli ST (heat-stable enterotoxin), and exotoxin A from Pseudomonas aeruginosa. Bacterial outer membrane proteins such as outer membrane complex c (OMPC), porins, transferrin binding proteins, pneumococcal surface protein A (PspA; See International Application Patent Publication No. WO 02/091998), pneumococcal adhesin protein (PsaA), C5a peptidase from Group A or Group B streptococcus, or Haemophilus influenzae protein D, pneumococcal pneumolysin (Kuo et al., 1995, Infect Immun 63; 2706-13) including ply detoxified in some fashion for example dPLY-GMBS (See International Patent Application Publication No. WO 04/081515) or dPLY-formol, PhtX, including PhtA, PhtB, PhtD, PhtE and fusions of Pht proteins for example PhtDE fusions, PhtBE fusions (See International Patent Application Publication Nos. WO 01/98334 and WO 03/54007), can also be used. Other proteins, such as ovalbumin, keyhole limpet hemocyanin (KLH), bovine serum albumin (BSA) or purified protein derivative of tuberculin (PPD), PorB (from N. meningitidis), PD (Haemophilus influenzae protein D; see, e.g., European Patent No. EP 0 594 610 B), or immunologically functional equivalents thereof, synthetic peptides (See European Patent Nos. EP0378881 and EP0427347), heat shock proteins (See International Patent Application Publication Nos. WO 93/17712 and WO 94/03208), pertussis proteins (See International Patent Application Publication No. WO 98/58668 and European Patent No. EP0471177), cytokines, lymphokines, growth factors or hormones (See International Patent Application Publication No. WO 91/01146), artificial proteins comprising multiple human CD4+ T cell epitopes from various pathogen derived antigens (See Falugi et al., 2001, Eur J Immunol 31:3816-3824) such as N19 protein (See Baraldoi et al., 2004, Infect Immun 72:4884-7), iron uptake proteins (See International Patent Application Publication No. WO 01/72337), toxin A or B of C. difficile (See International Patent Publication No. WO 00/61761), and flagellin (See Ben-Yedidia et al., 1998, Immunol Lett 64:9) can also be used as carrier proteins.

Other DT mutants can be used as the second carrier protein, such as CRM₁₇₆, CRM₂₂₈, CRM₄₅ (Uchida et al., 1973, J Biol Chem 218:3838-3844); CRM₉, CRM₄₅, CRM₁₀₂, CRM₁₀₃ and CRM₁₀₇ and other mutations described by Nicholls and Youle in Genetically Engineered Toxins, Ed: Frankel, Maecel Dekker Inc., 1992; deletion or mutation of Glu-148 to Asp, Gln or Ser and/or Ala 158 to Gly and other mutations disclosed in U.S. Pat. No. 4,709,017 or 4,950,740; mutation of at least one or more residues Lys 516, Lys 526, Phe 530 and/or Lys 534 and other mutations disclosed in U.S. Pat. No. 5,917,017 or 6,455,673; or fragment disclosed in U.S. Pat. No. 5,843,711.

Where multivant vaccines are used, a second carrier can be used for one or more of the antigens in a multivalent vaccine. The second carrier protein is preferably a protein that is non-toxic and non-reactogenic and obtainable in sufficient amount and purity. The second carrier protein is also conjugated or joined with an antigen, e.g., a S. pneumoniae polysaccharide to enhance immunogenicity of the antigen. Carrier proteins should be amenable to standard conjugation procedures. In one embodiment, each capsular polysaccharide not conjugated to the first carrier protein is conjugated to the same second carrier protein (e.g., each capsular polysaccharide molecule being conjugated to a single carrier protein). In another embodiment, the capsular polysaccharides not conjugated to the first carrier protein are conjugated to two or more carrier proteins (each capsular polysaccharide molecule being conjugated to a single carrier protein). In such embodiments, each capsular polysaccharide of the same serotype is typically conjugated to the same carrier protein.

### Multivalent Polysaccharide-Protein Conjugate Vaccines

In certain embodiments, the immunogenic compositions can comprise capsular polysaccharides from S. pneumoniae serotype selected from at least one of 1, 2, 3, 4, 5, 6A, 6B, 6C, 6D, 7B, 7C, 7F, 8, 9N, 9V, 10A, 11A, 12F, 14, 15A, 15B, 15C, 16F, 17F, 18C, 19A, 19F, 20, 21, 22A, 22F, 23A, 23B, 23F, 24F, 27, 28A, 31, 33F, 34, 35A, 35B, 35F, and 38 conjugated to one or more carrier proteins. Preferably, saccharides from a particular serotype are not conjugated to more than one carrier protein.

After the individual glycoconjugates are purified according to the methods of the invention, they are compounded to formulate a multivalent immunogenic composition. These pneumococcal conjugates are prepared by separate processes and bulk formulated into a single dosage formulation. While it is preferable that each conjugate include a surfactant for fiterabilty, one or more polysaccharide protein conjugates in the multivalent immunogenic composition can be prepared without surfactant.

### Pharmaceutical/Vaccine Compositions

In certain embodiments, compositions, including pharmaceutical, immunogenic and vaccine compositions, are provided comprising, consisting essentially of, or alternatively, consisting of any of the polysaccharide serotype combinations described above together with a pharmaceutically acceptable carrier and an adjuvant. In one embodiment, the compositions comprise, consist essentially of, or consist of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 distinct polysaccharide-protein conjugates, wherein each of the conjugates contains a different capsular polysaccharide conjugated to either a single carrier protein or a first and second carrier protein, and wherein the capsular polysaccharides from at least one of serotypes 1, 2, 3, 4, 5, 6A, 6B, 6C, 6D, 7B, 7C, 7F, 8, 9N, 9V, 10A, 11A, 12F, 14, 15A, 15B, 15C, 16F, 17F, 18C, 19A, 19F, 20, 21, 22A, 22F, 23A, 23B, 23F, 24F, 27, 28A, 31, 33F, 34, 35A, 35B, 35F, and 38 of Streptococcus pneumoniae are conjugated to CRM₁₉₇.

Formulation of the polysaccharide-protein conjugates of the present invention can be accomplished using art-recognized methods. For instance, 15 individual pneumococcal conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

In a preferred embodiment, the vaccine composition is formulated in L-histidine buffer with sodium chloride.

As defined herein, an “adjuvant” is a substance that serves to enhance the immunogenicity of an immunogenic composition of the invention. An immune adjuvant may enhance an immune response to an antigen that is weakly immunogenic when administered alone, e.g., inducing no or weak antibody titers or cell-mediated immune response, increase antibody titers to the antigen, and/or lowers the dose of the antigen effective to achieve an immune response in the individual. Thus, adjuvants are often given to boost the immune response and are well known to the skilled artisan. Suitable adjuvants to enhance effectiveness of the composition include, but are not limited to:

(1) aluminum salts (alum), such as aluminum hydroxide, aluminum phosphate, aluminum sulfate, etc.;

(2) oil-in-water emulsion formulations (with or without other specific immunostimulating agents such as muramyl peptides (defined below) or bacterial cell wall components), such as, for example, (a) MF59 (International Patent Application Publication No. WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton, Mass.), (b) SAF, containing 10% Squalene, 0.4% Tween 80, 5% pluronic-blocked polymer L121, and thr-MDP either microfluidized into a submicron emulsion or vortexed to generate a larger particle size emulsion, (c) Ribi™ adjuvant system (RAS), (Corixa, Hamilton, Mont.) containing 2% Squalene, 0.2% Tween 80, and one or more bacterial cell wall components from the group consisting of 3-O-deaylated monophosphorylipid A (MPL™) described in U.S. Pat. No. 4,912,094, trehalose dimycolate (TDM), and cell wall skeleton (CWS), preferably MPL+CWS (Detox™); and (d) a Montanide ISA;

(3) saponin adjuvants, such as Quil A or STIMULON™ QS-21 (Antigenics, Framingham, Mass.) (see, e.g., U.S. Pat. No. 5,057,540) may be used or particles generated therefrom such as ISCOM (immunostimulating complexes formed by the combination of cholesterol, saponin, phospholipid, and amphipathic proteins) and Iscomatrix® (having essentially the same structure as an ISCOM but without the protein);

(4) bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-tetradecanoyloxytetradecanoylamino]ethyl 2-Deoxy-4-O-phosphono-3-O—[(R)-3-tetradecanoyloxytetradecanoyl]-2-[(R)-3-tetradecanoyloxytetradecanoylamino]-b-D-glucopyranoside, which is also known as 529 (formerly known as RC529), which is formulated as an aqueous form or as a stable emulsion;

(5) synthetic polynucleotides such as oligonucleotides containing CpG motif(s) (U.S. Pat. No. 6,207,646);

(6) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc; and

(7) complement, such as a trimer of complement component C3d.

In another embodiment, the adjuvant is a mixture of 2, 3, or more of the above adjuvants, e.g., SBAS2 (an oil-in-water emulsion also containing 3-deacylated monophosphoryl lipid A and QS21).

Muramyl peptides include, but are not limited to, N-acetyl-muramyl-L-threonyl-D-isoglutamine (thr-MDP), N-acetyl-normuramyl-L-alanine-2-(1′-2′ dipalmitoyl-sn-glycero-3-hydroxyphosphoryloxy)-ethylamine (MTP-PE), etc.

In certain embodiments, the adjuvant is an aluminum salt. The aluminum salt adjuvant may be an alum-precipitated vaccine or an alum-adsorbed vaccine. Aluminum-salt adjuvants are well known in the art and are described, for example, in Harlow, E. and D. Lane (1988; Antibodies: A Laboratory Manual Cold Spring Harbor Laboratory) and Nicklas, W. (1992; Aluminum salts. Research in Immunology 143:489-493). The aluminum salt includes, but is not limited to, hydrated alumina, alumina hydrate, alumina trihydrate (ATH), aluminum hydrate, aluminum trihydrate, Alhydrogel®, Superfos, Amphogel®, aluminum (III) hydroxide, aluminum hydroxyphosphate sulfate (Aluminum Phosphate Adjuvant (APA)), amorphous alumina, trihydrated alumina, or trihydroxyaluminum.

APA is an aqueous suspension of aluminum hydroxyphosphate. APA is manufactured by blending aluminum chloride and sodium phosphate in a 1:1 volumetric ratio to precipitate aluminum hydroxyphosphate. After the blending process, the material is size-reduced with a high-shear mixer to achieve a monodisperse particle size distribution. The product is then diafiltered against physiological saline and steam sterilized.

In certain embodiments, a commercially available Al(OH)₃ (e.g. Alhydrogel® or Superfos of Denmark/Accurate Chemical and Scientific Co., Westbury, N.Y.) is used to adsorb proteins in a ratio of 50-200 g protein/mg aluminum hydroxide. Adsorption of protein is dependent, in another embodiment, on the pI (Isoelectric pH) of the protein and the pH of the medium. A protein with a lower pI adsorbs to the positively charged aluminum ion more strongly than a protein with a higher pI. Aluminum salts may establish a depot of Ag that is released slowly over a period of 2-3 weeks, be involved in nonspecific activation of macrophages and complement activation, and/or stimulate innate immune mechanisms (possibly through stimulation of uric acid). See, e.g., Lambrecht et al., 2009, Curr Opin Immunol 21:23.

Monovalent bulk aqueous conjugates are typically blended together and diluted to target 8 μg/mL for all serotypes except 6B, which will be diluted to target 16 μg/mL. Once diluted, the batch will be filter sterilized, and an equal volume of aluminum phosphate adjuvant added aseptically to target a final aluminum concentration of 250 μg/mL. The adjuvanted, formulated batch will be filled into single-use, 0.5 mL/dose vials.

In certain embodiments, the adjuvant is a CpG-containing nucleotide sequence, for example, a CpG-containing oligonucleotide, in particular, a CpG-containing oligodeoxynucleotide (CpG ODN). In another embodiment, the adjuvant is ODN 1826, which may be acquired from Coley Pharmaceutical Group.

“CpG-containing nucleotide,” “CpG-containing oligonucleotide,” “CpG oligonucleotide,” and similar terms refer to a nucleotide molecule of 6-50 nucleotides in length that contains an unmethylated CpG moiety. See, e.g., Wang et al., 2003, Vaccine 21:4297. In another embodiment, any other art-accepted definition of the terms is intended. CpG-containing oligonucleotides include modified oligonucleotides using any synthetic internucleoside linkages, modified base and/or modified sugar.

Methods for use of CpG oligonucleotides are well known in the art and are described, for example, in Sur et al., 1999, J Immunol. 162:6284-93; Verthelyi, 2006, Methods Mol Med. 127:139-58; and Yasuda et al., 2006, Crit Rev Ther Drug Carrier Syst. 23:89-110.

### Administration/Dosage

The compositions and formulations described herein can be used to protect or treat a human susceptible to infection, e.g., a pneumococcal infection, by means of administering the vaccine via a systemic or mucosal route. In one embodiment, the present invention provides a method of inducing an immune response to a S. pneumoniae capsular polysaccharide conjugate, comprising administering to a human an immunologically effective amount of an immunogenic composition of the present invention. In another embodiment, the present invention provides a method of vaccinating a human against a pneumococcal infection, comprising the step of administering to the human an immunogically effective amount of an immunogenic composition of the present invention.

Optimal amounts of components for a particular vaccine can be ascertained by standard studies involving observation of appropriate immune responses in subjects. For example, in another embodiment, the dosage for human vaccination is determined by extrapolation from animal studies to human data. In another embodiment, the dosage is determined empirically.

“Effective amount” of a composition of the invention refers to a dose required to elicit antibodies that significantly reduce the likelihood or severity of infectivitiy of a microbe, e.g., S. pneumonia, during a subsequent challenge.

The composition provided herein can be used for the prevention and/or reduction of primary clinical syndromes caused by microbes, e.g., S. pneumonia, including both invasive infections (meningitis, pneumonia, and bacteremia), and noninvasive infections (acute otitis media, and sinusitis).

Administration of the compositions can include one or more of: injection via the intramuscular, intraperitoneal, intradermal or subcutaneous routes; or via mucosal administration to the oral/alimentary, respiratory or genitourinary tracts. In one embodiment, intranasal administration is used for the treatment of pneumonia or otitis media (as nasopharyngeal carriage of pneumococci can be more effectively prevented, thus attenuating infection at its earliest stage).

The amount of conjugate in each vaccine dose is selected as an amount that induces an immunoprotective response without significant, adverse effects. Such amount can vary depending upon the pneumococcal serotype. Generally, for polysaccharide-based conjugates, each dose will comprise 0.1 to 100 μg of each polysaccharide, particularly 0.1 to 10 μg, and more particularly 1 to 5 μg. For example, each dose can comprise 100, 150, 200, 250, 300, 400, 500, or 750 ng or 1, 1.5, 2, 3, 4, 5, 6, 7, 7.5, 8, 9, 10, 11, 12, 13, 14, 15, 16, 18, 20, 22, 25, 30, 40, 50, 60, 70, 80, 90, or 100 μg.

Optimal amounts of components for a particular vaccine can be ascertained by standard studies involving observation of appropriate immune responses in subjects. For example, in another embodiment, the dosage for human vaccination is determined by extrapolation from animal studies to human data. In another embodiment, the dosage is determined empirically.

In one embodiment, the dose of the aluminum salt is 10, 15, 20, 25, 30, 50, 70, 100, 125, 150, 200, 300, 500, or 700 μg, or 1, 1.2, 1.5, 2, 3, 5 mg or more. In yet another embodiment, the dose of alum salt described above is per μg of recombinant protein.

In one embodiment, the subject is human. In certain embodiments, the human patient is an infant (less than 1 year of age), toddler (approximately 12 to 24 months), or young child (approximately 2 to 5 years). In other embodiments, the human patient is an elderly patient (>65 years). The compositions are also suitable for use with older children, adolescents and adults (e.g., aged 18 to 45 years or 18 to 65 years).

In one embodiment, a composition is administered as a single inoculation. In another embodiment, the vaccine is administered twice, three times or four times or more, adequately spaced apart. For example, the composition may be administered at 1, 2, 3, 4, 5, or 6 month intervals or any combination thereof. The immunization schedule can follow that designated for pneumococcal vaccines. For example, the routine schedule for infants and toddlers against invasive disease caused by S. pneumoniae is 2, 4, 6 and 12-15 months of age. Thus, in a preferred embodiment, the composition is administered as a 4-dose series at 2, 4, 6, and 12-15 months of age.

The compositions may also include one or more proteins from S. pneumoniae. Examples of S. pneumoniae proteins suitable for inclusion include those identified in International Patent Application Publication Nos. WO 02/083855 and WO 02/053761.

### Formulations

The compositions can be administered to a subject by one or more methods known to a person skilled in the art, such as parenterally, transmucosally, transdermally, intramuscularly, intravenously, intra-dermally, intra-nasally, subcutaneously, intra-peritonealy, and formulated accordingly.

In one embodiment, compositions are administered via epidermal injection, intramuscular injection, intravenous, intra-arterial, subcutaneous injection, or intra-respiratory mucosal injection of a liquid preparation. Liquid formulations for injection include solutions and the like.

The compositions can be formulated as single dose vials, multi-dose vials or as pre-filled syringes.

In another embodiment, compositions of the present invention are administered orally, and are thus formulated in a form suitable for oral administration, i.e., as a solid or a liquid preparation. Solid oral formulations include tablets, capsules, pills, granules, pellets and the like. Liquid oral formulations include solutions, suspensions, dispersions, emulsions, oils and the like.

Pharmaceutically acceptable carriers for liquid formulations are aqueous or nonaqueous solutions, suspensions, emulsions or oils. Examples of nonaqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

The pharmaceutical composition may be isotonic, hypotonic or hypertonic. However it is often preferred that a pharmaceutical composition for infusion or injection is essentially isotonic, when it is administrated. Hence, for storage the pharmaceutical composition may preferably be isotonic or hypertonic. If the pharmaceutical composition is hypertonic for storage, it may be diluted to become an isotonic solution prior to administration.

The isotonic agent may be an ionic isotonic agent such as a salt or a non-ionic isotonic agent such as a carbohydrate. Examples of ionic isotonic agents include but are not limited to NaCl, CaCl₂, KCl and MgCl₂. Examples of non-ionic isotonic agents include but are not limited to mannitol, sorbitol and glycerol.

It is also preferred that at least one pharmaceutically acceptable additive is a buffer. For some purposes, for example, when the pharmaceutical composition is meant for infusion or injection, it is often desirable that the composition comprises a buffer, which is capable of buffering a solution to a pH in the range of 4 to 10, such as 5 to 9, for example 6 to 8.

The buffer may for example be selected from the group consisting of TRIS, acetate, glutamate, lactate, maleate, tartrate, phosphate, citrate, carbonate, glycinate, histidine, glycine, succinate and triethanolamine buffer.

The buffer may furthermore for example be selected from USP compatible buffers for parenteral use, in particular, when the pharmaceutical formulation is for parenteral use. For example the buffer may be selected from the group consisting of monobasic acids such as acetic, benzoic, gluconic, glyceric and lactic; dibasic acids such as aconitic, adipic, ascorbic, carbonic, glutamic, malic, succinic and tartaric; polybasic acids such as citric and phosphoric; and bases such as ammonia, diethanolamine, glycine, triethanolamine, and TRIS.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, glycols such as propylene glycols or polyethylene glycol, Polysorbate 80 (PS-80), Polysorbate 20 (PS-20), and Poloxamer 188 (P188) are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

Generally, the surfactants used during filtration are carried over to the final formulation. However, the surfactant can also be removed using known methods and the formulation reconstituted in solutions containing the same or different surfactant.

Thus, the formulations may also contain a surfactant. Preferred surfactants include, but are not limited to: the polyoxyethylene sorbitan ester surfactants (commonly referred to as the Tweens), especially PS-20 and PS-80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. A preferred surfactant for including in the emulsion is PS-80.

Mixtures of surfactants can be used, e.g. PS-80/Span 85 mixtures. A combination of a polyoxyethylene sorbitan ester such as polyoxyethylene sorbitan monooleate (PS-80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% w/v) are: polyoxyethylene sorbitan esters (such as PS-80) 0.01 to 1% (w/v), in particular about 0.1% (w/v); octyl- or nonylphenoxy polyoxyethanols (such as Triton X-100, or other detergents in the Triton series) 0.001 to 0.1% (w/v), in particular 0.005 to 0.02% (w/v); polyoxyethylene ethers (such as laureth 9) 0.1 to 20% (w/v), preferably 0.1 to 10% (w/v) and in particular 0.1 to 1% (w/v) or about 0.5% (w/v).

In certain embodiments, the composition consists essentially of histidine (20 mM), saline (150 mM) and 0.02% PS-20 or 0.04% PS-80 at a pH of 5.8 with 250 ug/mL of APA (Aluminum Phosphate Adjuvant). PS-20 can range from 0.005% to 0.1% (w/v) with the presence of PS-20 or PS-80 in formulation controlling aggregation during simulated manufacture and in shipping using primary packaging. Process consists of combining blend of up to 24 serotypes in histidine, saline, and PS-20 or PS-80 then combining this blended material with APA and saline with or without antimicrobial preservatives.

As demonstrated herein, the choice of surfactant may need to be optimized for different drug products and drug substances. For multivalent vaccines having 15 or more serotypes, PS-20 and P188 are preferred. The choice of chemistry used to make conjugate can also play an important role in the stabilization of the formulation. In particular, as shown in the Examples, when the conjugation reactions used to prepare different polysaccharide protein conjugates in a multivalent composition include both aqueous chemistry and DMSO chemistry, it has been found that particular surfactant systems provide significant differences in stability.

Surfactant is included in the methods of the invention to produce polysaccharide protein conjugates. Such surfactant is typically carried over into the formulation of the drug product. However, the amount of surfactant carrier over is generally lower than desired for the final formulation. Accordingly, additional surfactant is generally added to obtain the desired concentration.

In certain embodiments, the surfactant system comprises polysorbate 20 (IUPAC name: Polyoxyethylene (20) sorbitan monolaurate; PS-20), a commercially available surfactant, commonly referred to as the Tween® 20. In certain embodiments, the final concentration of the polysorbate 20 in the formulations of the invention is in the range from 0.001% to 10% (w/v), from 0.025% to 2.5% (w/v), or 0.025% to 0.1% (w/v). A surfactant system comprising polysorbate 20 may further comprise a polyol. The polyol may be selected from propylene glycol and polyethylene glycol. In certain aspects, the polyethylene glycol or propylene glycol is at a final concentration of 6% to 20% (w/v). In certain aspects, the polyethylene glycol is polyethylene glycol 400.

In certain embodiments, the surfactant system comprises a poloxamer having a molecular weight in the range from 1100 Da to 17,400 Da and a polyol selected from propylene glycol and polyethylene glycol 400.

A poloxamer is a nonionic triblock copolymer composed of a central hydrophobic chain of polyoxypropylene (poly(propylene oxide)) flanked by two hydrophilic chains of polyoxyethylene (poly(ethylene oxide)). Poloxamers are also known by the tradename Pluronic®. Because the lengths of the polymer blocks can be customized, many different poloxamers exist that have slightly different properties. For the generic term “poloxamer”, these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the Pluronic® tradename, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobe; and the last digit×10 gives the percentage polyoxyethylene content (e.g., L61=Pluronic® with a polyoxypropylene molecular mass of 1,800 g/mol and a 10% polyoxyethylene content). See U.S. Pat. No. 3,740,421.

Examples of poloxamers have the general formula:

HO(C₂H₄O)ₐ(C₃H₆O)_{b}(C₂H₄O)ₐH,

wherein a and b blocks have the following values:

| Pluronic ® | Poloxamer | a | b | Molecular Weight |
|---|---|---|---|---|
| L31 | | 2 | 16 | 1100 (average) |
| L35 | | | | 1900 (average) |
| L44NF | 124 | 12 | 20 | 2090 to 2360 |
| L64 | | | | 2900 (average) |
| L81 | | | | 2800 (average) |
| L121 | | | | 4400 (average) |
| P123 | | 20 | 70 | 5750 (average) |
| F68NF | 188 | 80 | 27 | 7680 to 9510 |
| F87NF | 237 | 64 | 37 | 6840 to 8830 |
| F108NF | 338 | 141 | 44 | 12700 to 17400 |
| F127NF | 407 | 101 | 56 | 9840 to 14600 |

Molecular weight units, as used herein, are in Dalton (Da) or g/mol.

For the formulations, a poloxamer generally has a molecular weight in the range from 1100 Da to 17,400 Da, from 7,500 Da to 15,000 Da, or from 7,500 Da to 10,000 Da. The poloxamer can be selected from poloxamer 188 or poloxamer 407. The final concentration of the poloxamer in the formulations of the invention is from 0.001% to 5% weight/volume, or 0.025% to 1% weight/volume.

The formulation also contains a pH-buffered saline solution. The buffer may, for example, be selected from the group consisting of TRIS, acetate, glutamate, lactate, maleate, tartrate, phosphate, citrate, carbonate, glycinate, histidine, glycine, succinate, HEPES (4-(2-hydroxyethyl)-1-piperazineethanesulfonic acid), MOPS (3-(N-morpholino)propanesulfonic acid), MES (2-(N-morpholino)ethanesulfonic acid) and triethanolamine buffer. The buffer is capable of buffering a solution to a pH in the range of 4 to 10, 5.2 to 7.5, or 5.8 to 7.0. In certain aspects of the invention, the buffer is selected from the group consisting of phosphate, succinate, histidine, MES, MOPS, HEPES, acetate or citrate. The buffer may furthermore, for example, be selected from USP compatible buffers for parenteral use, in particular, when the pharmaceutical formulation is for parenteral use. The concentrations of buffer will range from 1 mM to 50 mM or 5 mM to 50 mM. In certain aspects, the buffer is histidine at a final concentration of 5 mM to 50 mM, or succinate at a final concentration of 1 mM to 10 mM. In certain aspects, the histidine is at a final concentration of 20 mM±2 mM.

While the saline solution (i.e., a solution containing NaCl) is preferred, other salts suitable for formulation include but are not limited to, calcium chloride (CaCl₂), potassium chloride (KCl) and magnesium chloride (MgCl₂) and combinations thereof. Non-ionic isotonic agents including but not limited to sucrose, trehalose, mannitol, sorbitol and glycerol may be used in lieu of a salt. Suitable salt ranges include, but are not limited to, 25 mM to 500 mM or 40 mM to 170 mM. In one aspect, the saline is sodium chloride (NaCl), optionally present at a concentration from 20 mM to 170 mM.

In a preferred embodiment, the formulations comprise a L-histidine buffer with sodium chloride.

In certain embodiments of the formulations described herein, the polysaccharide-protein conjugates comprise one or more pneumococcal polysaccharides conjugated to a carrier protein. The carrier protein can be selected from CRM₁₉₇, diphtheria toxin fragment B (DTFB), DTFB C8, Diphtheria toxoid (DT), tetanus toxoid (TT), fragment C of TT, pertussis toxoid, cholera toxoid, E. coli LT, E. coli ST, exotoxin A from Pseudomonas aeruginosa, and combinations thereof. In one aspect, all of the polysaccharide-protein conjugates are prepared using aqueous chemistry. In another aspect, one or more of the polysaccharide protein conjugates are prepared using DMSO chemistry. As an example, the polysaccharide-protein conjugate formulation can be a 15-valent pneumococcal conjugate formulation wherein polysaccharide protein conjugates from serotypes 6A, 6B, 7F, 18C, 19A, 19F, and 23F are prepared using DMSO chemistry and polysaccharide protein conjugates from serotypes 1, 3, 4, 5, 9V, 14, 22F, and 33F are prepared using aqueous chemistry.

In another embodiment, the pharmaceutical composition is delivered in a controlled release system. For example, the agent can be administered using intravenous infusion, a transdermal patch, liposomes, or other modes of administration. In another embodiment, polymeric materials are used; e.g. in microspheres in or an implant.

The compositions of this invention may also include one or more proteins from S. pneumoniae. Examples of S. pneumoniae proteins suitable for inclusion include those identified in International Patent Application Publication Nos. WO 02/083855 and WO 02/053761.

Having described various embodiments of the invention with reference to the accompanying description and drawings, it is to be understood that the invention is not limited to those precise embodiments, and that various changes and modifications may be effected therein by one skilled in the art without departing from the scope or spirit of the invention as defined in the appended claims.

The following examples illustrate, but do not limit the invention.

### EXAMPLES

### Example 1: Preparation of S. Pneumoniae Capsular Polysaccharides

Methods of culturing pneumococci are well known in the art. See, e.g., Chase, 1967, Methods of Immunology and Immunochemistry 1:52. Methods of preparing pneumococcal capsular polysaccharides are also well known in the art. See, e.g., European Patent No. EP0497524. Isolates of pneumococcal subtypes are available from the American Type Culture Collection (Manassas, Va.). The bacteria are identified as encapsulated, non-motile, Gram-positive, lancet-shaped diplococci that are alpha-hemolytic on blood-agar. Subtypes can be differentiated on the basis of Quelling reaction using specific antisera. See, e.g., U.S. Pat. No. 5,847,112.

Cell banks representing each of the S. pneumococcus serotypes present were obtained from the Merck Culture Collection (Rahway, N.J.) in a frozen vial. A thawed seed culture was transferred to the seed fermentor containing a pre-sterilized growth media appropriate for S. pneumoniae. The culture was grown in the seed fermentor with temperature and pH control. The entire volume of the seed fermentor was transferred to a production fermentor containing pre-sterilized growth media. The production fermentation was the final cell growth stage of the process. Temperature, pH, and the agitation rate were controlled.

The fermentation process was terminated via the addition of an inactivating agent. After inactivation, the batch was transferred to the inactivation tank where it was held at controlled temperature and agitation. Cell debris was removed using a combination of centrifugation and filtration. The batch was ultrafiltered and diafiltered. The batch was then subjected to solvent-based fractionations that remove impurities and recover polysaccharide.

### Example 2: Conjugation of Serotypes 1, 3, 4, 5, 6A, 6B, 7F, 9V, 14, 18C, 19A, 19F, 22F, 23F, and 33F to CRM₁₉₇ Using Reductive Amination in Aqueous Solution

The different serotype polysaccharides were individually conjugated to the purified CRM₁₉₇ carrier protein using a common process flow. Polysaccharide was dissolved, size reduced, chemically activated and buffer-exchanged by ultrafiltration. Purified CRM₁₉₇ was then conjugated to the activated polysaccharide as described in Example 3 except with NiCl₂ (2 mM), and the resulting conjugate was purified by ultrafiltration prior to a final 0.2 micron filtration. Several process parameters within each step, such as pH, temperature, concentration, and time were controlled to serotype-specific values as shown below.

### Polysaccharide Size Reduction and Oxidation

Purified pneumococcal capsular Ps powder was dissolved in water, and all serotypes, except serotype 19A, were 0.45-micron filtered. All serotypes, except serotype 19A, were homogenized to reduce the molecular mass of the Ps. Serotype 19A was not size reduced due to its relatively low starting size. Homogenization pressure and number of passes through the homogenizer were controlled to serotype-specific targets (150-1000 bar; 4-7 passes) to achieve a serotype-specific molecular mass. Size-reduced polysaccharide was 0.2-micron filtered and then concentrated and diafiltered against water using a 10 kDa NMWCO (Nominal Molecular Weight Cut Off) tangential flow ultrafiltration membrane.

The polysaccharide solution was then adjusted to a serotype-specific temperature (4-22° C.) and pH (4-5) with a sodium acetate buffer to minimize Ps size reduction due to activation. For all serotypes (except serotype 4), Ps activation was initiated with the addition of a 100 mM sodium metaperiodate solution. The amount of sodium metaperiodate added was serotype-specific, ranging from approximately 0.1 to 0.5 moles of sodium metaperiodate per mole of polysaccharide repeating unit. The serotype-specific charge of sodium metaperiodate was to achieve a target level of Ps activation (moles aldehyde per mole of Ps repeating unit). For serotype 4, prior to the sodium metaperiodate addition, the batch was incubated at approximately 50° C. and pH 4.1 to partially deketalize the Ps. This partial deketalization of the serotype 4 polysaccharide was completed prior to the addition of sodium metaperiodate.

For all serotypes, with the exception of serotypes 5 and 7F, the activated product was diafiltered against 10 mM potassium phosphate, pH 6.4 using a 10 kDa NMWCO tangential flow ultrafiltration membrane. Serotypes 5 and 7F were diafiltered against 10 mM sodium acetate, pH 4-5. Ultrafiltration for all serotypes was conducted at 2-8° C.

### Polysaccharide Conjugation to CRM₁₉₇

Oxidized polysaccharide solution was blended with water and 1.5 M potassium phosphate, pH 6.0 or pH 7.0, depending on the serotype. The selected buffer pH was to improve the stability of activated Ps during the conjugation reaction. Purified CRM₁₉₇, obtained through expression in Pseudomonas fluorescens as previously described (See International Patent Application Publication No. WO 2012/173876 A1), was 0.2-micron filtered and combined with the buffered polysaccharide solution at a polysaccharide to CRM₁₉₇ mass ratio ranging from 0.4 to 1.0 w/w depending on the serotype. The mass ratio was selected to control the polysaccharide to CRM₁₉₇ ratio in the resulting conjugate. The polysaccharide and phosphate concentrations were serotype-specific, ranging from 3.6 to 10.0 g/L and 100 to 150 mM, respectively, depending on the serotype. The serotype-specific Ps concentration was selected to control the size of the resulting conjugate. The solution was then 0.2-micron filtered. Nickel chloride was added to approximately 2 mM using a 100 mM nickel chloride solution. Sodium cyanoborohydride (2 moles per mole of polysaccharide repeating unit) was added. Conjugation proceeded for a serotype-specific duration (72 to 120 hours) for consumption of Ps and protein.

Reduction with Sodium Borohydride

Following the conjugation reaction, the batch was diluted to a Ps concentration of approximately 3.5 g/L, cooled to 2-8° C., and 1.2-micron filtered. All serotypes (except serotype 5) were diafiltered against 100 mM potassium phosphate, pH 7.0 at 2-8° C. using a 100 kDa NMWCO tangential flow ultrafiltration membrane. The batch, recovered in the retentate, was then diluted to approximately 2.0 g Ps/L and pH-adjusted with the addition of 1.2 M sodium bicarbonate, pH 9.4. Sodium borohydride (1 mole per mole of polysaccharide repeating unit) was added. After 6 hours, 1.5 M potassium phosphate, pH 6.0 was added. Serotype 5 was diafiltered against 300 mM sodium bicarbonate, pH 9, using a 100 kDa NMWCO tangential flow ultrafiltration membrane.

### Final Filtration and Product Storage

The batch was then concentrated and diafiltered against 10 mM histidine in 150 mM sodium chloride, pH 7.0 at 4° C. using a 300 kDa NMWCO tangential flow ultrafiltration membrane. The retentate batch was 0.2 micron-filtered.

Serotype 19F was incubated for approximately 7 days, diafiltered against 10 mM histidine in 150 mM sodium chloride, pH 7.0 at 4° C. using a 100 kDa NMWCO tangential flow ultrafiltration membrane, and 0.2-micron filtered.

The batch was adjusted to a Ps concentration of 1.0 g/L with additional 10 mM histidine in 150 mM sodium chloride, pH 7.0. The batch was dispensed into aliquots and frozen at ≤−60° C.

### Example 3: Methods for the Conjugation of Serotypes 6A, 7F, 18C, 19A, 19F, and 23F to CRM₁₉₇ Using Reductive Amidation in Dimethylsulfoxide

The different serotype polysaccharides 6A, 7F, 18C, 19A, 19F, and 23F were individually conjugated to the purified CRM₁₉₇ carrier protein using a common process flow. Polysaccharide was dissolved, sized to a target molecular mass, chemically activated and buffer-exchanged by ultrafiltration. Activated polysaccharide and purified CRM₁₉₇ were individually lyophilized and redissolved in dimethylsuloxide (DMSO). Redissolved polysaccharide and CRM₁₉₇ solutions were then combined and conjugated as described below. The resulting conjugate was purified by ultrafiltration prior to a final 0.2-micron filtration. Several process parameters within each step, such as pH, temperature, concentration, and time were controlled to serotype-specific values in section below.

### Polysaccharide Size Reduction and Oxidation

Purified pneumococcal capsular Ps powder was dissolved in water, and all serotypes, except serotype 19A, were 0.45-micron filtered. All serotypes, except serotypes 18C and 19A, were homogenized to reduce the molecular mass of the Ps. Homogenization pressure and number of passes through the homogenizer were controlled to serotype-specific targets (150-1000 bar; 4-7 passes). Serotype 18C was size-reduced by acid hydrolysis at ≥90° C.

Size-reduced polysaccharide was 0.2-micron filtered and then concentrated and diafiltered against water using a 10 kDa NMWCO tangential flow ultrafiltration membrane. A 5 kDa NMWCO membrane was used for serotype 18C.

The polysaccharide solution was then adjusted to a serotype-specific temperature (4-22° C.) and pH (4-5) with a sodium acetate buffer. Ps activation was initiated with the addition of a 100 mM sodium metaperiodate solution. The amount of sodium metaperiodate added was serotype-specific, ranging from approximately 0.1 to 0.3 moles of sodium metaperiodate per mole of polysaccharide repeating unit.

For all serotypes, the activated product was diafiltered against 10 mM potassium phosphate, pH 6.4 using a 10 kDa NMWCO tangential flow ultrafiltration membrane. A 5 kDa NMWCO membrane was used for serotype 18C. Ultrafiltration for all serotypes was conducted at 2-8° C.

### Polysaccharide Conjugation to CRM₁₉₇

Purified CRM₁₉₇, obtained through expression in Pseudomonas fluorescens as previously described (WO 2012/173876 A1), was diafiltered against 2 mM phosphate, pH 7.0 buffer using a 5 kDa NMWCO tangential flow ultrafiltration membrane and 0.2-micron filtered.

The oxidized polysaccharide solution was formulated with water and sucrose in preparation for lyophilization. The protein solution was formulated with water, phosphate buffer, and sucrose in preparation for lyophilization. Sucrose concentrations ranged from 1 to 5% to achieve optimal redissolution in DMSO following lyophilization.

Formulated Ps and CRM₁₉₇ solutions were individually lyophilized. Lyophilized Ps and CRM₁₉₇ materials were redissolved in DMSO and combined using a tee mixer. Sodium cyanoborohydride (1 mole per mole of polysaccharide repeating unit) was added, and conjugation proceeded for a serotype-specific duration (1 to 48 hours) to achieve a targeted conjugate size.

Reduction with Sodium Borohydride

Sodium borohydride (2 moles per mole of polysaccharide repeating unit) was added following the conjugation reaction. The batch was diluted into 150 mM sodium chloride at approximately 4° C. Potassium phosphate buffer was then added to neutralize the pH. The batch was concentrated and diafiltered at approximately 4° C. against 150 mM sodium chloride using a 30 kDa NMWCO tangential flow ultrafiltration membrane.

### Final Filtration and Product Storage

Each batch was then concentrated and diafiltered against 10 mM histidine in 150 mM sodium chloride, pH 7.0 at 4° C. using a 300 kDa NMWCO tangential flow ultrafiltration membrane. The retentate batch was 0.2-micron filtered.

Serotype 19F was incubated for approximately 5 days, diafiltered against 10 mM histidine in 150 mM sodium chloride, pH 7.0 at approximately 4° C. using a 300 kDa NMWCO tangential flow ultrafiltration membrane, and 0.2-micron filtered.

The batch was diluted with additional 10 mM histidine in 150 mM sodium chloride, pH 7.0 and dispensed into aliquots and frozen at ≤−60° C.

### Ultrafiltration Membranes

The type of ultrafiltration membrane was investigated to determine whether it would have an effect on the yield. Conjugates as prepared in this example were diafiltered against 10 mM histidine in 150 mM sodium chloride, pH 7.0 buffer containing 0.03% w/v PS-20 using membranes of regenerated cellulose and polyethersulfone.

As shown in Table 1, the recovery of a serotype 6A-CRM₁₉₇ conjugate (Lot 1), when diafiltered against buffer containing PS-20, was +18% higher when the 300 kD membrane was composed of regenerated cellulose compared to polyethersulfone. Similarly, the recovery of a serotype 6B-CRM197 conjugate (Lot 2) was +20% higher when diafiltered using a 300 kD regenerated cellulose membrane compared to a 300 kD polyethersulfone membrane.

TABLE 1
Recovery of polysaccharides using diafiltration
with different membrance materials

| | | Material of | |
|---|---|---|---|
| | PS-20 content | construction of | |
| | in diafiltration | 300 kD NMCWO | Conjugated |
| Conjugate Lot | buffer (% w/v) | Membrane | Ps yield (%) |
| Serotype | 0.03 | Polyethersulfone | 70 |
| 6A-CRM197 | 0.03 | Regenerated | 88 |
| Conjugate Lot 1 | | cellulose | |
| Serotype | 0.03 | Polyethersulfone | 68 |
| 6B-CRM197 | 0.03 | Regenerated | 88 |
| Conjugate Lot 2 | | cellulose | |

In the presence of PS-20, the material of construction of the membrane used for diafiltration was surprisingly found to impact significantly the recovery of the conjugate.

### Example 4: Use of Surfactants to Improve the Filterability of Conjugates

Larger Ps-protein conjugates have been shown in the literature to be more immunogenic than smaller conjugates (See, e.g., Lee et al. 2009, Vaccine. 27(5): 726-732). However, the ability of the commonly used 0.2-micron filter to filter Ps-protein conjugate becomes more challenging as conjugate size increases.

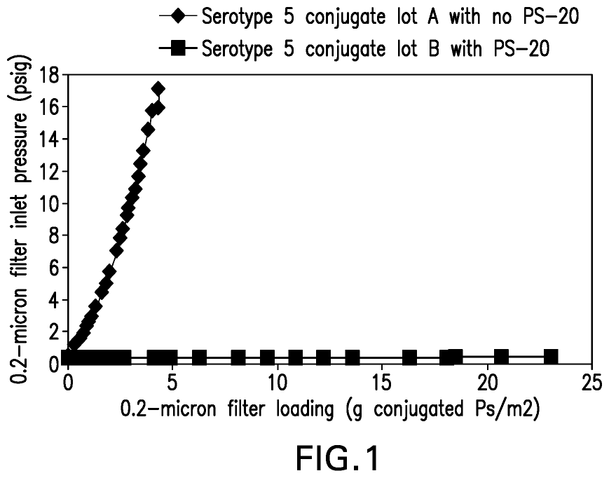

Conjugate filtration studies were performed to investigate the effect of PS-20 surfactant on the filterability of large Ps-CRM₁₉₇ conjugates. Two serotype 5-CRM₁₉₇ conjugates (lots A and B, produced using the aqueous conjugation process described in Example 2) were 0.2-micron filtered at constant flow rate. Both lots were in 10 mM histidine, 150 mM sodium chloride, pH 7 buffer. Lot A did not contain PS-20. Lot B contained PS-20 by diafiltration into 10 mM histidine, 150 mM sodium chloride, pH 7 buffer with 0.05% (w/v) PS-20. Conjugate size of lot B was significantly larger (+21% larger) than lot A as measured by HPSEC/UV/MALS/RI. Filter inlet pressure rise as a function of mass loading (grams of conjugated Ps per m² of filter area) for lots A and B is shown in FIG. 1. The sharp pressure rise for lot A indicated that lot A was significantly less filterable, despite having a lower conjugate size, than lot B. Lot B showed no measurable pressure rise during the filtration.

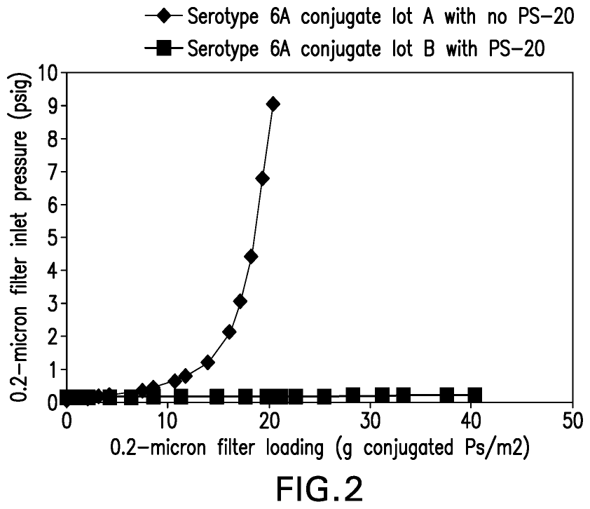

A similar study was performed using two serotype 6A-conjugates to demonstrate that the filtration improvement with PS-20 can be extended to other serotypes as well as using a different solvent. Serotype 6A lots A and B were produced using the DMSO conjugation process described in Example 3. Lots were filtered at constant flow rate through a 0.2-micron filter. Both lots were in 10 mM histidine, 150 mM sodium chloride, pH 7 buffer. Lot A did not contain PS-20. Lot B contained PS-20 by diafiltration into 10 mM histidine, 150 mM sodium chloride, pH 7 buffer with 0.03% (w/v) PS-20. Conjugate size of lot B was significantly larger (+34% larger) than lot A as measured by HPSEC/UV/MALS/RI. Similar to the results shown in FIG. 1, the sharp pressure rise for lot A in FIG. 2 indicated that lot A was less filterable despite having a lower conjugate size than lot B. Lot B showed no measurable pressure rise during the filtration.

### Molecular Weight and Concentration Analysis of Conjugates Using HPSEC/UV/MALS/RI Assay

Conjugate samples were injected and separated by high performance size-exclusion chromatography (HPSEC). Detection was accomplished with ultraviolet (UV), multi-angle light scattering (MALS) and refractive index (RI) detectors in series. Protein concentration was calculated from UV280 using an extinction coefficient. Polysaccharide concentration was deconvoluted from the RI signal (contributed by both protein and polysaccharide) using the do/dc factors which are the change in a solution's refractive index with a change in the solute concentration reported in mL/g. Average molecular weight of the samples were calculated by Astra software (Wyatt Technology Corporation, Santa Barbara, Calif.) using the measured concentration and light scattering information across the entire sample peak.

### Example 5: Protective Effect of Surfactants Against Aggregation of Pneumococcal Polysaccharide-CRM₁₉₇ DMSO Conjugates

A screening method to evaluate the tendency of pneumococcal polysaccharide-CRM₁₉₇ conjugates to aggregate was developed. Conjugates made in aqueous conditions were prepared as described in Example 2. Conjugates made under DMSO conditions were prepared as described in Example 3.

In an initial experiment, serotype 23F-CRM₁₉₇ conjugates, prepared as described in Example 3, were subjected to a brief vortexing stress and then evaluated by flow cytometry. PS-20 was spiked to one set of samples to final concentration of 0.05% (w/v), while the other set had no PS-20. Individual particles of aggregated pneumococcal polysaccharide serotype 23F-CRM₁₉₇ conjugates passing through the flow cell were detected by light scattering. Particle concentration and the intensity of scattered light, which is proportional to their size and refractive index, were used to evaluate the degree of aggregation. Flow cytometry has been used as a rapid tool to evaluate aggregation in biological samples (See, Mach et al., 2011, J. Pharm. Sci. 100(5):1671-8). Although flow cytometers are not sensitive enough to detect monomeric conjugates they can be used to detect aggregated molecules.

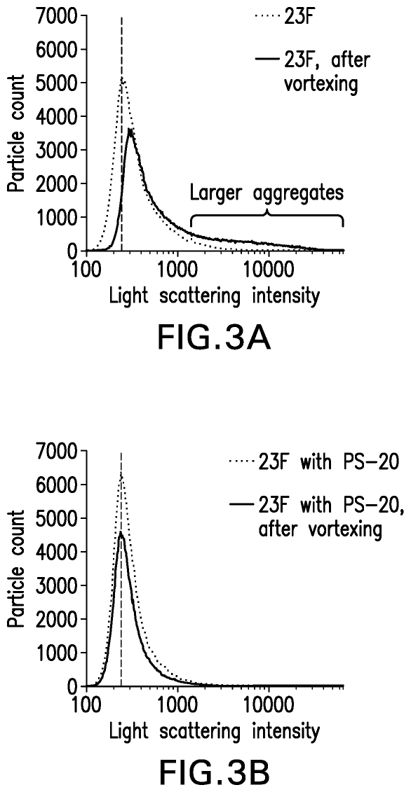

Proteins or protein conjugates may aggregate and/or precipitate when surface agitation or shear forces are introduced. This phenomenon can be observed during routine processing or handling like pumping, mixing, ultrafiltration/diafiltration, stirring, membrane filtration, filling, transportation, etc. Vortexing with head space air is one of the common approaches to induce surface and shear denaturation. Therefore, vortexing with fixed power and time was chosen to produce consistent physical stress condition. Vortexing of conjugates often resulted in the appearance of particles with significantly larger light scatter signal, which suggests particles of a larger size (FIG. 3).

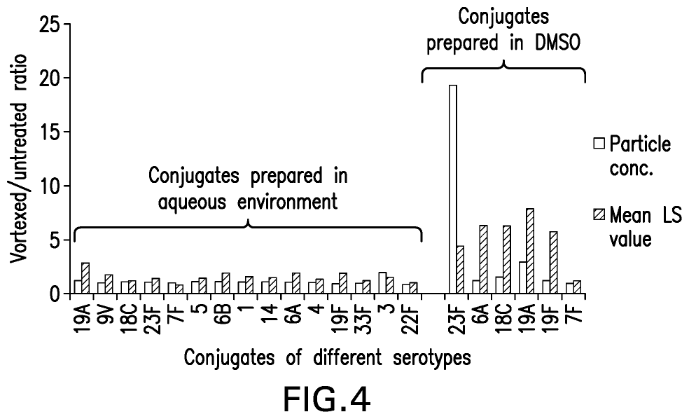

A broader set of conjugates were prepared under aqueous condition or under DMSO conditions and subjected to vortexing stress and flow cytometry as described above. The ratio of vortexed to untreated was determined for each serotype for particles/μl and mean LS value. Conjugates prepared in the presence of DMSO were found to be more prone to aggregation than conjugates prepared under aqueous conditions (FIG. 4) as determine by mean LS value and particles/μl.

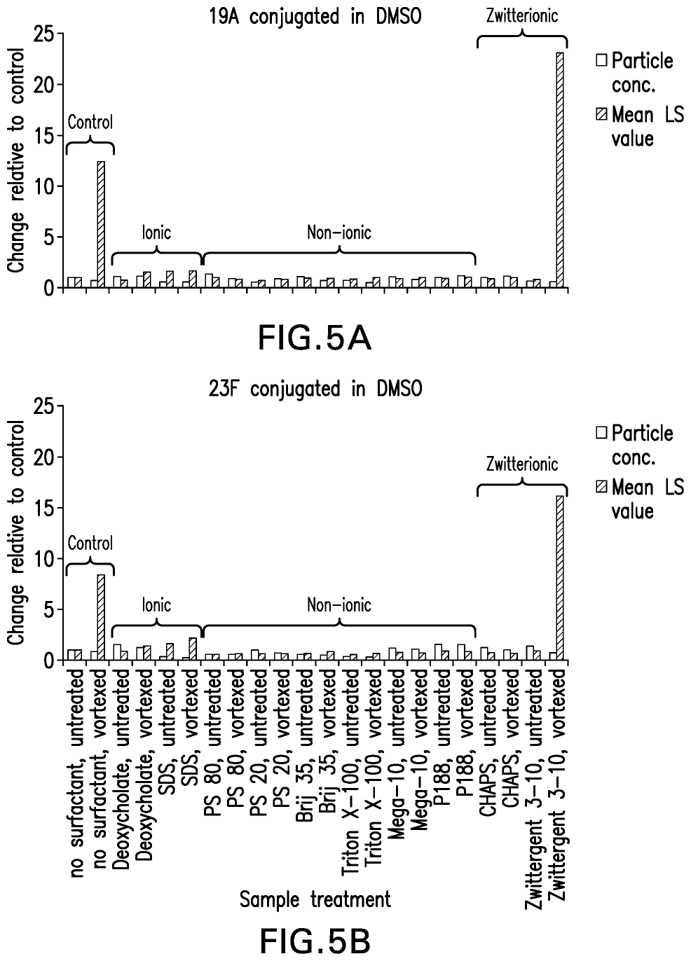

A panel of surfactants was tested for their ability to alleviate aggregation using the methods described above. Serotypes 19A and 23F prepared, as described in Example 3, were selected as representative serotypes for the screen. Various surfactants (ionic surfactants: deoxycholate, sodium dodecyl sulfate (SDS); nonionic surfactants: PS-20, PS-80, Brij 35, Triton X-100, Mega-10, and P188; zwitterionic surfactants: CHAPS, Zwittergent 3-10) were added to the sample to a final concentration of 0.05% (w/v). The samples were then measured by flow cytometry with or without vortexing. With the exception of Zwittergent 3-10, the addition of the surfactant, including ionic, non-ionic and zwitterionic, prevented the accumulation of larger particles upon vortexing (FIGS. 3 and 5).

1. A method for purifying a polysaccharide protein conjugate, the method comprising:
a) reacting a polysaccharide with a protein to form a polysaccharide protein conjugate reaction mixture;
b) performing purification of the polysaccharide protein conjugate reaction mixture by ultrafiltration, diafiltration or column chromatography; and
c) performing a sterile filtration of the polysaccharide protein conjugate reaction mixture in the presence of a surfactant.

2. The method of claim 1, wherein the surfactant is added during step c).

3. The method of claim 1, wherein the surfactant is added during step b).

4. The method of claim 1, wherein the surfactant is added during step a).

5. The method of claim 1, wherein the sterile filtration is by a 0.2-0.5 μm filter.

6. The method of claim 5, wherein the sterile filtration is by sterilizing or bioburden reduction filtration using a 0.2 μm or 0.22 μm filter.

7. The method of claim 1, wherein the surfactant is an ionic surfactant, a nonionic surfactant or a zwitterionic surfactant.

8. The method of claim 7, wherein the zwitterionic surfactant is CHAPS.

9. The method of claim 1, wherein the surfactant is a nonionic surfactant.

10. The method of claim 9, wherein the surfactant is PS-20 present at a concentration range between 0.007% to 0.5% (w/v), inclusive.

11. The method of claim 1, wherein the purifying in step b) is by ultrafiltration or diafiltration.

12. The method of claim 11, wherein the ultrafiltration or diafiltration is performed using a 50 to 1,000 kDa MWCO membrane made of regenerated cellulose.

13. The method of claim 12, wherein the the ultrafiltration or diafiltration filter is performed using a 300 kDa MWCO membrane made of regenerated cellulose.

14. The method according to claim 1, wherein the polysaccharide is selected from the group consisting of Meningococcal polysaccharides, Pneumococcal polysaccharides, Hemophilus influenzae type b polysaccharide, Vi polysaccharide of Salmonnella typhi, and group B Streptococcus polysaccharides.

15. The method of claim 14, wherein the polysaccharide is a pneumococcal polysaccharide.

16. The method according to claim 1, wherein the protein is selected from the group consisting of tetanus toxoid, diphtheria toxoid, and CRM₁₉₇.

17. The method of claim 16, wherein the protein is CRM₁₉₇.

18. The method of claim 1, wherein said reacting is by reductive amination.

19. The method of claim 18, wherein the reductive amination is performed under aqueous conditions.

20. The method of claim 18, wherein the reductive amination is performed in dimethylsulfoxide (DMSO).

21. The method of claim 18, wherein said reductive amination comprises:
a) reacting a polysaccharide with an oxidizing agent, whereby a solution of an aldehyde-activated polysaccharide is obtained;
b) adjusting the pH of the solution of the aldehyde-activated polysaccharide to a pH of from 4 to 7, if necessary; and
c) reacting the activated polysaccharide with a protein in the presence of a reducing agent at a pH of from 6 to 7.5 to form a polysaccharide protein conjugate.

22. The method of claim 21, further comprising reacting the polysaccharide protein conjugate with a strong reducing agent.

23. The method of claim 21, wherein the oxidizing agent is sodium periodate (NaIO₄).

24. The method according to claim 21, wherein the solution of the aldehyde-activated polysaccharide is buffer exchanged with a buffer containing acetate or phosphate.

25. The method according to claim 21, wherein the reducing agent is sodium cyanoborohydride (NaCNBH₃) or sodium borohydride (NaBH₄).

26. The method of claim 22, wherein the strong reducing agent is NaBH₄.

27. The method of claim 1 comprising:
a) reacting a pneumococcal polysaccharide with a protein by reductive amination to form a polysaccharide protein conjugate reaction mixture;
b) performing purification of the polysaccharide protein conjugate reaction mixture by ultrafiltration; and
c) performing a sterile filtration using a 0.2 μm or 0.22 μm filter of the polysaccharide protein conjugate reaction mixture in the presence of PS-20 present at a concentration range between 0.007% to 0.5% (w/v).
